(19) United States
(12) Patent Application Publication
(10) Pub. No.: US 2008/0275546 A1
(43) Pub. Date: Nov. 6, 2008

(54) Inhibitory cell adhesion surfaces

(75) Inventors: Daniel M. Storey, Minneapolis, MN (US); Luke J. Ryves, Minneapolis, MN (US); Barbara S. Kitchell, Holmes Beach, FL (US)

Correspondence Address: CHAMELEON SCIENTIFIC CORPORATION;AKA IONIC FUSION CORPORATION, 13355 10TH AVENUE NORTH, SUITE 108, PLYMOUTH, MN, 55441, US

(73) Assignee: Chameleon Scientific Corp

(21) Appl. No.: 12/148,971

(22) Filed: Apr. 24, 2008

Related U.S. Application Data
(60) Provisional application No. 60/927,353, filed on May 3, 2007.

(51) Int. Cl. A61F 2/82; H05H 1/24; A61F 2/24; A61F 2/12; A61F 2/30; A61F 2/02

(52) U.S. Cl. 623/1.46; 427/576; 623/2.42; 623/8; 623/18.11; 424/423

(57) ABSTRACT

Textured nanostructured surfaces are described which are highly resistant to cell adhesion. Such surfaces on medical implants inhibit fibroblast adhesion particularly on titanium treated silicone. The surfaces can also be engineered so that other cell types, such as endothelial and osteoblast cells, show little if any tendency to attach to the surface in vivo.

This application claims benefit of U.S. provisional application Ser. No. 60/927,353 filed May 3, 2007.

### BACKGROUND OF THE INVENTION

1. Field of the Invention

The invention relates generally to the field of engineered surfaces, particularly to surfaces modified for increased resistance to cell adhesion.

2. Description of Background Art

Inhibition of cell adhesion on various surfaces is a particularly important goal in the design of certain medical devices, particularly those devices where obstruction or cell proliferation is undesirable. Such devices used in vivo are susceptible to undesirable cell adherence and proliferation. Implants, vascular prostheses and kidney dialysis equipment are especially prone to undesirable overgrowths of soft tissue cells such as fibroblasts, resulting in failure of the device and need for short term replacement.

Studies on surface modifications are typically designed to identify materials that enhance cell adhesion; for example, enhancement of osteointegration on implanted titanium alloys or RGD-coated titanium implants (Elmengaard, et al., 2005). Bioactive proteins such as collagen and fibronectin have been attached to dental implants in order to enhance gingival fibroblast binding and enhance sealing of soft tissue to implant surfaces.

Yet cell adhesion and proliferation remains a concern for many types of implants and devices used in contact with tissue or body fluids. For example, coatings have been employed on intraocular lenses in order to lessen damage to endothelial cells when the lenses are inserted as well as to mitigate formation of biofilms. Different plastic coatings deposited from a plasma reactor onto the lens have been used to provide defined thickness coatings of selected polymers on poly(methylacrylate). Film materials included perfluoropropane, ethylene oxide, 2-hydroxyethyl methacrylate and N-vinyl-2-pyrrolidone (Mateo and Ratner, 1989).

In efforts to prevent cells from adhering to glass surfaces, Owens, et al. (1987) studied a large number of polymers coated on glass for ability to prevent adhesion by red blood cells, Dictyostelium discoideum amoebae and Escherichia coli. Polyethylene oxide (PEO) for example was already known to have anti-adhesive properties used either alone or as a co-polymer, as demonstrated by lack of adhesion of platelets and rabies virus on coated glass. The researchers tested several co-polymer coatings on glass using polymers that had hydrophobic and hydrophilic segments. The three types of cells tested in vitro were readily washed from a hydrophobic glass surface coated with a bifunctional F-106 Pluronic, demonstrating lack of adhesion even after 1 hr exposure to the cells.

More recently Ishihara, et al. (1999) studied fibroblast adhesion and proliferation on polymer coated poly(ethylene terephthalate) substrates. Cell adherence appeared to be related to the hydrophobicity of the coated surface, in turn determined by the composition of the co-polymer. Polymers poly 2-methacryloyloxyethyl phosphorylcholine (MPC)-co-n-butyl methacrylate copolymer and poly(2-hydroxyethylmethacrylate) were tested. Higher amounts of MPC in the one copolymer led to a weakening of the interaction between the polymer surface and adhering proteins and consequently a decrease in the number of fibroblast cells adhering to those surfaces.

Medical devices and implants that remain in the body for any period of time tend to act as foci of inflammation, due in part to adherence and build up of fibrous tissue when fibroblasts proliferate on the surfaces of orthopedic or vascular implants. There is a need for methods of modifying surface characteristics of materials used in vivo so that undesirable cell attachment does not occur.

### SUMMARY OF THE INVENTION

The present invention concerns a process for treating surfaces to significantly reduce cell adhesion compared with the unmodified surface. The nonadherent surfaces are illustrated with several different substrate materials and with different types of cells, including fibroblasts, endothelial and osteoblast cells.

An important feature of the invention is the preparation of structured surfaces that effectively change surface energy and hydrophobicity. Surface energy can be increased so that cell adherence is significantly weakened. As disclosed herein, the described structured surfaces do not promote cell adhesion or, if adhered, will readily disengage from the surface; for example, in situations where laminar flow is involved such as on surfaces of medical implants exposed to blood flow in vivo.

In particular, it is shown that activated surfaces can be created on the surface of a selected substrate, metal or non-metal, thereby raising surface energy and significantly decreasing cell adhesion and proliferation. An example is the controlled titanium plasma treatment of a silicone surface. When fibroblast adhesion to the treated surface was tested, cell density was decreased over 50% compared with adhesion to untreated silicone surfaces. In contrast, treatment of polytetrafluoroethylene (PTFE) and ultra high molecular weight polyethylene (UHMWPE) substrates using different titanium plasma exposure conditions lowered rather than increased surface energy, resulting in up to a 180% increase in cell density on the treated surface compared with the untreated surface.

Surface energy can be increased or decreased for virtually any surface using a controlled plasma surface treatment procedure. Generally, this requires creating a plasma and controlling macromolecule deposition on a selected surface. The size and distribution of the macromolecules determines surface energy and hydrophobicity of the surface. In effect, an ion plasma treatment method (IPD) can be used to increase surface area on selected substrates. This results in higher surface energy, increased hydrophobicity and decreased cell adherence compared to untreated surfaces.

Surfaces of bone and vascular implants are particularly susceptible to in vivo cell adhesion. Materials currently used for medical implants include titanium, titanium alloys such as Ti6Al4V and CoCrMo alloys, silicone, polyethylene and the like. The surface treatments disclosed herein can be adapted to texturize a substrate surface so that cell adhesion is significantly reduced.

Changes in surface characteristics as a result of using the disclosed surface treatment were assessed by measuring the dynamic contact angle. Increased or decreased contact angles on treated surfaces were exhibited by water droplets depending on the treatment conditions. Treatment of a silicone surface with plasma generated titanium nanoparticles caused increased water contact angles with the surface. On the other hand, selectively modifying the titanium generated plasma exposure on UHMWPE and PTFE resulted in decreased water contact angles with the treated surface compared with untreated surfaces. When water droplet contact angles were decreased, there was increased cell adhesion and proliferation on the surfaces.

### BRIEF DESCRIPTION OF THE FIGURES

FIG. 1 shows decreased fibroblast adhesion on a titanium treated silicone (Si) surface compared with UHMWPE and PTFE titanium treated and untreated (Si—C, UHMWPE-C and PTFE-C) surfaces. +=p<0.01 compared with corresponding untreated sample.

FIG. 2A shows fibroblast proliferation measured as increased cell density on titanium treated silicone, UHMWPE and PTFE after one day in vitro exposure to fibroblast cells compared with untreated surfaces.

FIG. 2B shows fibroblast proliferation measured as increased cell density on titanium treated silicone, UHMWPE and PTFE after three days in vitro exposure to fibroblast cells compared with untreated surfaces.

FIG. 2C shows fibroblast proliferation measured as increased cell density on titanium treated silicone, UHMWPE and PTFE after five days in vitro exposure to fibroblast cells compared with untreated surfaces.

FIG. 3 shows fluorescent images comparing fibroblast proliferation on a titanium plasma treated and untreated silicone, PE and PTFE surfaces. Cell counting used DAPI dye under fluorescent microscope. Fluorescing dots are cell nuclei. Uncoated silicone has a higher cell density compared to titanium coated silicone. Day 3 (or day 5) data.

FIG. 4 shows the general features of a modified cathodic arc IPD apparatus: target 1; substrate 2; movable substrate holder 3; vacuum chamber 4; power supply 5 for the target; and arc control 6 to adjust speed of the arc.

FIG. 5 shows the increased water droplet contact angles for silicone (Si), polyethylene (PE), and Teflon® (PTFE) for untreated surfaces and for titanium plasma treated surfaces (Si—C, PE-C and PTFE-C).

### DETAILED DESCRIPTION OF THE INVENTION

The present invention provides controlled nano-textured surfaces particularly suitable for medical implant surfaces where in vivo cell adhesion is undesirable. A surface treatment has been developed that has the ability to decrease the attachment of osteoblast, endothelial, and fibroblast cells to the treated surfaces compared to cell attachment on the untreated surface.

By exposing a surface to an activated plasma that can be adjusted to change hydrophobic characteristics of a surface, the surface energy of a substrate can be raised. This results in the inhibition of attachment of various types of cells, in contrast to literature reported observations that the lowering of surface energy generally leads to an increase in cell attachment.

In order to provide surfaces that inhibit cell adhesion inhibition, a selected metal or polymer surface is exposed to a plasma such as titanium produced by an IPD process under defined conditions. Surface hydrophobicity is increased in a manner that appears to be related to the size of ion particulates on the substrate surface and the resulting surface texturing. The treatment conditions can be adjusted to control the size of nanoparticles that contact and texture the surface. It is believed that the nanoparticle treatment increases the surface area and raises surface energy, thereby increasing hydrophobicity and significantly decreasing cell adherence.

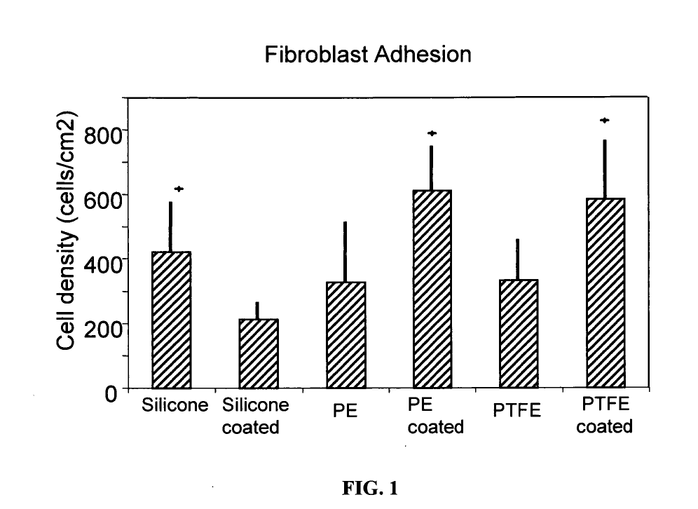

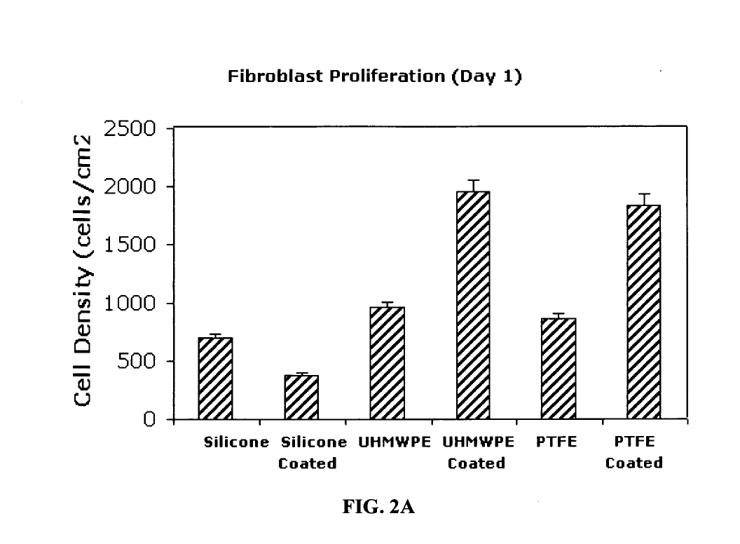

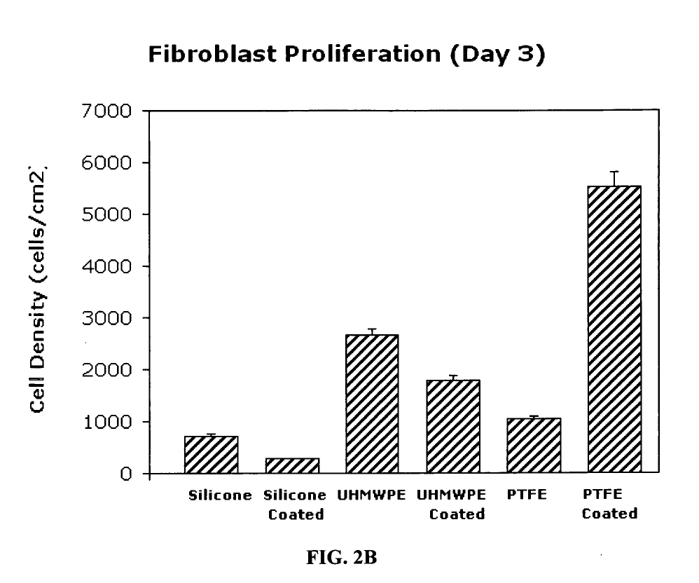

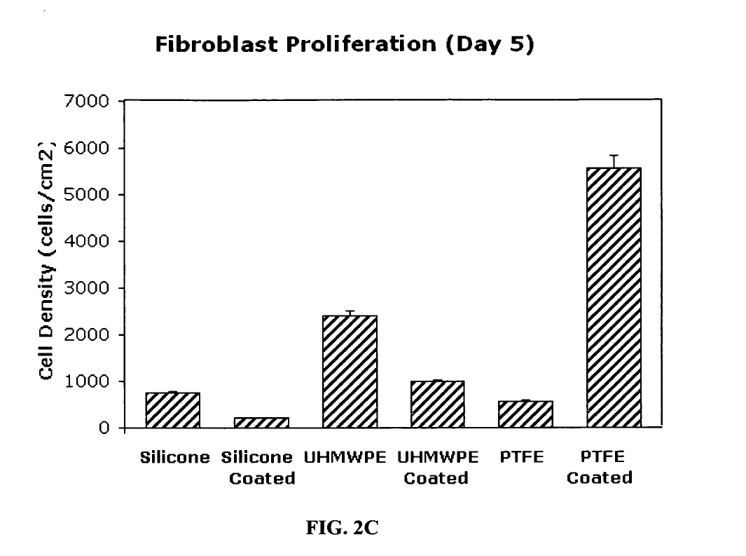

Effects on surface energy have been demonstrated for various materials in relation to the surface treatment. As seen in FIG. 1, a silicone surface was treated so that the surface energy of the surface was increased, while a modification of the plasma treatment conditions for UHMWPE and PTFE resulted in a decrease in surface energy compared with the untreated surfaces. Significantly decreased fibroblast density after 1, 3 and 5 days on the treated silicone surface was observed in contrast with the highly increased cell densities observed on treated UHMWPE and PTFE surfaces as shown in FIGS. 2A, 2B and 2C.

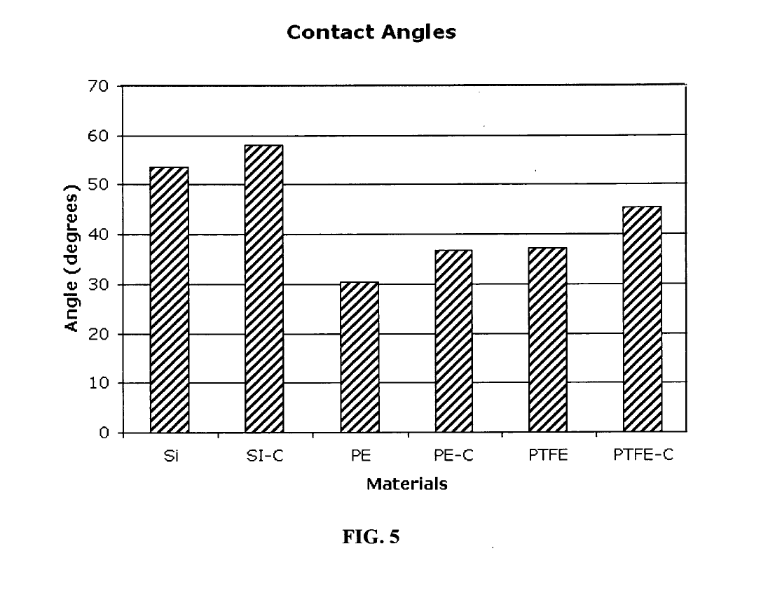

Measurement of the contact angle on the treated silicone, UHMWPE and PTFE surfaces using a water droplet showed that the contact angle increased on the silicone surface but decreased on the other treated surfaces compared with the respective untreated surfaces. An increase in contact angle indicates an increase in surface energy, and thus the hydrophobicity. The decrease in hydrophobicity on the UHMWPE and PTFE surfaces correlates with the increased cell adhesion on those surfaces compared with the decreased adhesion observed on the treated silicone surface. FIG. 5 shows the contact angle measurements for a water droplet on treated and untreated silicone, UHMWPE and PTFE surfaces.

The texturing and treatment of the different substrate surfaces utilized a titanium ion plasma deposition (IPD) process. This process creates nano-rough nanoparticulates on the surface of the substrates, thus changing the surface energy and creating a more hydrophobic surface. Basic procedures for creating a nano-rough surface can be found in Webster, et al. (2006).

Controlling the texturing of a wide range of materials using a customized IPD process provides control of the surface energy of any material. Because the surface treatment is independent of the substrate, this ability to control surface hydrophobicity and therefore cell adhesion characteristics will be applicable to any material.

The size of the nano texturing (i.e., particle size) directly controls the surface energy and the hydrophobicity. Thus, the IPD process can be adjusted to control the physical characteristics of the nano texturing so that in effect the surface energy of virtually any substrate can be engineered.

Materials

Fibroblasts (purchased from ATCC) were grown in culture until confluence in DMEM with 10% FBS and 1% P/S. Material samples were used as supplied. Before cell experiments, samples were sonicated and autoclaved.

Endothelial cells (purchased from ATCC) were grown in culture until confluence in DMEM with 10% FBS and 1% P/S. Material samples were used as supplied. Before cell experiments, samples were sonicated and autoclaved.

Osteoblasts (purchased from ATCC) were grown in culture until confluence in DMEM with 10% FBS and 1% P/S.

### EXAMPLES

The following examples are provided as illustrations of the invention and are in no way to be considered limiting.

### Example 1

### Ion Plasma Deposition

Ion Plasma Deposition (IPD) is a method of creating highly energized plasma using a cathodic arc discharge created from a target material, typically solid metal. An arc is struck on the metal and the high power density on the arc vaporizes and ionizes the metal, creating a plasma which sustains the arc. A vacuum arc is different from a high pressure arc because the metal vapor itself is ionized, rather than an ambient gas.

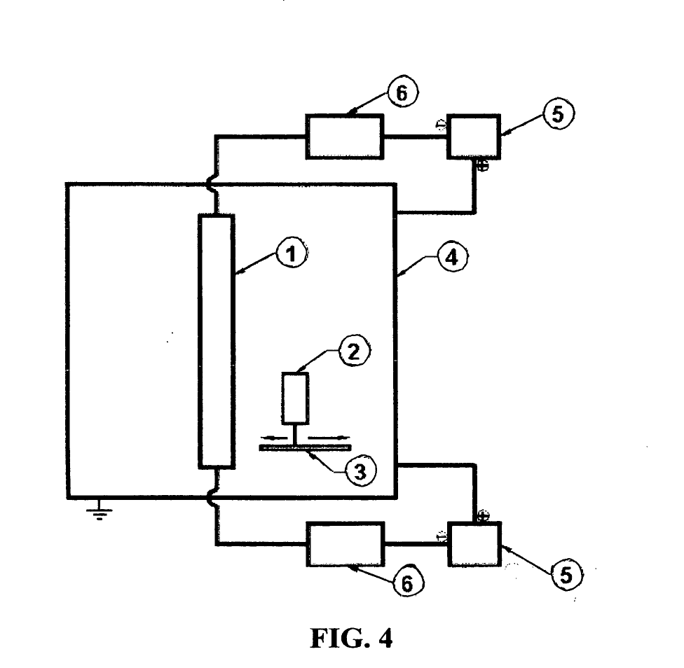

FIG. 4 illustrates an apparatus suitable for controlling deposition of the plasma ejected from the cathodic arc target source 1 onto a substrate 2. The size of the particle deposited, and thus the degree of nanotexturing of the deposited surface is controlled by a movable substrate holder 3 within the vacuum chamber 4 or by a power supply 5 to the target and adjustment of arc speed 6. The closer a substrate is to the arc source, the larger and more densely packed will be the particles deposited on the substrate.

Control of the substrate position with respect to the target and arc speed allow precise control of the surface characteristics of the substrate with respect to density, number and size of the nanoparticles arranged in the substrate surface. This in turn determines the surface area of the substrate and affects hydrophobic properties of the substrate surface. Hydrophobicity of a nanoparticle textured surface can be determined by measurement of the contact angle of a water droplet on the surface.

### Example 2

### Fibroblast Attachment/Repulsion

Three types of substrates were treated with Ti 6-4 using the IPD process to form a deposit with random depth up to 200 nm. The average nano-particle size of the coating was 10 to 30 nanometers and was confirmed by SEM analysis.

Fibroblasts were seeded onto each substrate at 3500 cells/cm². Samples were first placed in 12 and 24 well cell culture plates. 175 μl of cell-containing droplets in media were added to the samples before incubating at 37° C. and 5% CO₂ for 4 hours. The samples were washed 3 times with PBS, fixed in formaldehyde for 10 min, and again washed in PBS 3 times. Cells were then counted using fluorescent microscopy and DAPI dye. Images of cell morphology were also acquired. Experiments were conducted in triplicate with two repeats each (total of six samples for each averaged data point). Standard statistical analysis by Student t-test was used to determine differences between substrates.

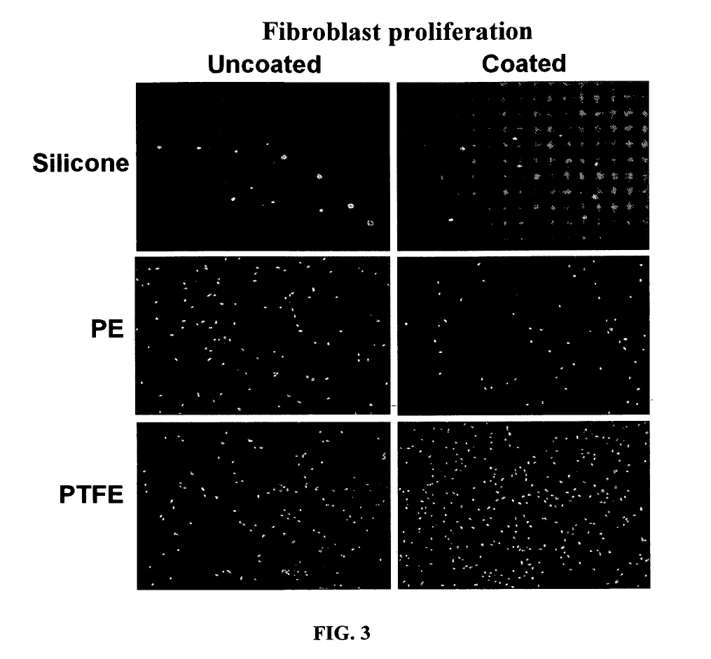

Results showed an unexpected decrease in in vitro fibroblast adhesion on titanium treated silicone compared to all other samples tested (FIG. 3) at one, three and five days after exposure to fibroblast cells. This suggested that less adhesion of fibroblasts will translate into less soft, scar tissue formation around either an orthopedic or vascular implant composed of titanium coated on silicone.

Qualitative fibroblast morphology images matched the quantitative data showing less fibroblast adhesion on titanium coated silicone. Specifically, less well-spread cells were observed on titanium coated silicone compared to other substrates tested.

### Example 3

### Decreased Endothelial Cell Adhesion on Titanium Coated Silicone

Silicone was treated with Ti 6-4 using the IPD process to form textured thicknesses up to 200 nm. The average nano-particle size of the coating was 30 to 40 nanometers and was confirmed by SEM analysis.

Endothelial cells were seeded onto each substrate at 3500 cells/cm². Samples were first placed in 12 and 24 well cell culture plates. 17511 of cell-containing droplets in media were added to the samples and were incubated at 37° C. and 5% CO₂ for 4 hours. The specimens were then washed 3 times with PBS, fixed in formaldehyde for 10 min, and again washed three times in PBS. Cells were then counted using fluorescent microscopy and DAPI dye. Images of cell morphology were also acquired. Experiments were conducted in triplicate with two repeats each (total of six samples for each averaged data point). Standard statistical analysis by Student t-test was used to determine differences between substrates.

Results showed a decrease in cell adhesion on the coated silicone parts of approximately 25%.

### Example 4

### Decreased Osteoblast Proliferation on Titanium-Coated Surfaces

Three types of substrates were treated with Ti 6-4 using the IPD process. The average nano-particle size on the surface was 10 to 30 nanometers and was confirmed by SEM analysis.

Purchased substrate samples were used as supplied. The samples were trimmed with a razor to make the adhesion surface flat. Before cell experiments, samples were sonicated in 70% ethanol and autoclaved or UV treated for 20 minutes.

Osteoblasts were seeded onto each substrate at 3500 cells/cm², then placed in 12 and 24 well cell culture plates. 175 μl of cell-containing droplets in media was added to the samples and then incubated at 37° C. and 5% CO₂ for 4 hours. At the end of the 4 hours the cell containing droplets were removed and each well with a sample filled with DMEM media and incubated again under the same conditions for 1, 3, and 5 day proliferation. Specimens were then washed 3 times with PBS, fixed in formaldehyde for 10 min, and again washed in PBS 3 times after 24, 72, and 120 hours respectively. Cells were counted using fluorescent microscopy and DAPI dye. Images of cell morphology were also be acquired. Experiments were conducted in triplicate with two repeats each (total of six samples for each averaged data point). Standard statistical analysis by Student t-test were used to determine differences between substrates.

Results of the 1, 3 and 5 day test are expected to show decreased osteoblast proliferation on all coated substrates over their uncoated counterparts as was shown for fibroblast cells (see FIG. 3).

### Example 5

### Decreased Cell Attachment Using IPD Surface Treatment

An IPD treatment was used to modify a silicone surface by employing a titanium plasma to create a nanoparticulate textured nano-roughness surface. The roughness characteristics of nanostructured titanium surfaces that enhance cell adherence have been reported (Webster, et al., 2004) but these surfaces, while produced from an ion plasma, are different in structure and physical characteristics from the treated surfaces prepared and tested in this example.

Several nano-structured titanium surfaces were prepared and tested for hydrophobicity and surface energy. Different types of cells were expected and did in fact show varying degrees of adhesion.

### Example 6

### Controlled Increase of Surface Energy

This example showed that controlled deposition of nanoparticles on selected surfaces will affect and can be used to change surface energy. As illustrated in FIG. 1 and FIGS. 2A-C, silicone treated with under IPD conditions to increase surface energy showed little tendency for fibroblast adherence, while UHMWPE and PTFE, each treated to lower surface energy, exhibited increased fibroblast adherence compared with the respective untreated surfaces.

In a 4 hr fibroblast adhesion assay, droplets containing 3500 cells/cm² were incubated on silicone, UHMWPE and PTFE titanium coated surfaces. After incubation, the samples were washed with PBS and the cells fixed with formaldehyde and stained with DAPI dye. Titanium treated UHMWPE and PTFE surfaces exposed in vitro to fibroblasts resulted in higher fibroblast densities on the treated surfaces compared to uncoated surfaces, while titanium treated silicone surfaces had a lower density of cell adhesion compared with the uncoated material. Data show a mean plus/minus standard deviation where *p<0.01 compared with the uncoated counterpart.

FIG. 5 compares surface contact angle of a water droplet on silicone, UHMWPE and PTFE surfaces treated with IPD titanium, showing that the surface treatment used on the silicone surface had a higher surface energy resulting in lower cell adherence as indicated by the increased contact angle on the silicone surface compared to the decreased contact angle for UHMWPE and PTFE relative to their uncoated surfaces.

### REFERENCES

Elmengaard, B., Bechtold, J. E. and Soballe, K., J., “In vivo effects of RGD-coated titanium implants inserted in two bone-gap models”, Biomedical Materials Research, Part A, v. 75A, (2), 249-255 (2005).
Mateo, N. B. and Ratner, B. D., “Relating the surface properties of intraocular lens materials to endothelial cell adhesion damage”, Investigative Opthalmology & Visual Science, v. 30 (5), May 1989, 853-860.
Owens, N. F., Gingell, D. and Rutter, “Inhibition of cell adhersion by a synthetic polymer adsorbed to glass shown under defined hydrodynamic stress”, P. R., J. Cell Sci. 87, 667-675 (1987)
Ishihara, K., Ishikawa, E., Iwasaki, Y. and Nakabayashi, N., “Inhibition of fibroblast cell adhesion on substrate by coating with 2-methacryloyloxyethyl phosphorylcholine polymers”, Biomater. Sci. Polym. Ed., 10(10), 1047-61 (1999)
Webster, et al. BSME Conference, Chicago, Ill., October 2006

What is claimed is:

1. A method for treating a selected target surface to provide increased surface hydrophobicity, comprising the steps:
exposing the target surface to an ionic plasma comprising activated metal ions;
selecting conditions to allow deposition of controlled size nanoparticles on the target surface;
wherein the deposited nanoparticles impart increased surface hydrophobicity compared to the target surface before exposure to the ionic plasma.

2. The method of claim 1 wherein the treated target surface has a higher surface energy than the untreated target surface.

3. The method of claim 1 wherein the selected target surface is a metal or polymer.

4. The method of claim 3 wherein the metal is titanium, Ti6Al4V or CoCrMo.

5. The method of claim 3 wherein the polymer is polyethylene, silicone, ultra high molecular weight polyethylene (UHMWPE), or polytetrafluoroethylene (PTFE).

6. A titanium implant device having a nanostructured high energy surface resistant to cell adhesion in vivo wherein the surface comprises sufficient number of ionic plasma deposited nanoparticles to increase hydrophobicity.

7. The implant device of claim 5 wherein the cell resistant to adhesion is a fibroblast, endothelial or osteoblast cell.

8. A method for altering the surface energy of a substrate surface, comprising:
depositing titanium nanoparticulates onto a selected surface by ionic plasma deposition (IPD); and
monitoring the hydrophobicity of the surface-deposited nanoparticulates;
wherein a change in surface hydrophobicity is indicative of an increase or decrease in surface energy compared with surface energy of the selected surface before deposition of titanium nanoparticles.

9. The method of claim 8 wherein the selected surface is polyethylene, silicone, UHMWPE, or PTFE.

10. The method of claim 8 wherein the selected surface is titanium or a titanium alloy.

11. The method of claim 8 wherein the ionic plasma target comprises titanium.

12. The method of claim 8 wherein the ionic plasma target comprises nitinol.

13. A medical device having a surface comprising an ionic plasma deposited (IPD) titanium hydrophobic coating inhibitory to cell adhesion.

14. The medical device of claim 13 wherein the surface of the medical device is silicone, ultra high molecular weight polyethylene (UHMWPE) or polytetrafluoroethylene (PTFE).

15. The medical device of claim 13 wherein a cell selected from the group consisting of fibroblasts, endothelial cells and osteoblasts are inhibited from adhering to the surface.

16. The medical device of claim 13 which is a stent, indwelling catheter, heart valve, polymer breast implants, joint implants, implanted leads for neural stimulation or pacemakers.
